(19) United States
(12) Patent Application Publication
(10) Pub. No.: US 2016/0058891 A1
(43) Pub. Date: Mar. 3, 2016

(54) IN VIVO ADCC MODEL

(71) Applicant: Roche Glycart AG, Schleren (CH)

(72) Inventors: Daniel Breustedt, Loerrach (DE); Christian Gerdes, Erlenbach (CH); Antonio Iglesias, Freiburg (DE); Pablo Umana, Wollerau (CH)

(73) Assignee: Roche Glycart AG, Schlieren (CH)

(21) Appl. No.: 14/773,637

(22) PCT Filed: Oct. 29, 2012
(86) PCT No.: PCT/EP2012/071328

§ 371 Date: Sep. 8, 2015

(30) Foreign Application Priority Data
Nov. 1, 2011 (EP) 11187392.3

(51) Int. Cl. A61K 49/00; A01K 67/027; G01N 33/50

(52) U.S. Cl. CPC A61K 49/0008; G01N 33/5011; A01K 67/0278; A01K 2267/0331; A01K 2207/15; A01K 2227/10

(57) ABSTRACT

The present invention relates to a non-human animal comprising a humanized low affinity FcgR locus. Also provided herein is the use of said non-human animal for determining in vivo efficacy of antibodies, and methods for determining in vivo efficacy of antibodies.

The field of invention is transgenic non-human animals that comprise a humanized low affinity Fc-γ receptor (FcgR) locus, including transgenic non-human animals that comprise a replacement of endogenous low affinity FcgR genes by human low affinity FcgR genes and including non-human animals that are capable of expressing four functional human low affinity FcgR genes, and including non-human animals that do not express endogenous low affinity FcgR genes.

Therapeutic antibodies (TherAbs) have proven highly efficacious for the treatment of human diseases such as neoplastic disorders or autoimmune inflammatory afflictions. Often, the therapeutic action of TherAbs is based on the cytotoxic ablation of tumor cells, such that the effector function properties of these antibody agents have become a crucial issue. TherAbs of murine origin often lead to immune reactions in the patients treated (anti-drug antibody (ADA) reaction). While purely human TherAbs are expected to substantially mitigate the issue of ADA reactions, the predictability of their biological efficacy before entering human treatment still remains largely unresolved. Due to the immense costs associated with the development of new TherAbs there is a need for tools to predict their efficacy early in that process. The in vivo potency of anti-tumor antibody drugs is commonly tested in xenograft assays. Hereby the target human tumor is first inoculated in immune deficient mice, typically Scid or RAG mutants, and allowed to grow, followed by the injection of the TherAbs. The efficacy of the TherAbs is determined by their capacity to eliminate the human tumor. In this system the destruction of the inoculated cancer cells relies largely on the activation of effector functions on (murine) cells of the native immune system by the introduced (human) TherAbs.

Binding of the Antigen-Antibody complex to Complement (C) or Fc-receptors (FcR) on the surface of different immune cells triggers the antibody dependent cell cytotoxicity (ADCC), whereby an effector cell of the immune system actively lyses a target (tumor) cell. Complement dependent cytotoxicity (CDC), macrophage-mediated phagocytosis or cytolysis as induced by Natural Killer cells (NK) or granulocytes; contribute to the destruction of cancer cells.

However, the efficiency of these mechanisms varies in different species and even in different individuals of the same species. In particular, while mice have only two activating low affinity FcR genes (Fcgr3 and Fcgr4), humans have four activating low affinity FcR genes (FCGR2A, FCGR2C, FCGR3A, FCGR3B). Moreover, the main functional human FcR, the FcγRIIIa (encoded by the FCGR3A gene), is found in NK cells and macrophages while its murine homolog FcγR4 is found in granulocytes and macrophages but not in NK cells. Human granulocytes express FCGR3B, an FcR with no counterpart in mice. Finally, the low affinity mouse Fcg3 is expressed in macrophages, NK cells and granulocytes and its human homolog FcgR2a is only found in macrophages and granulocytes while the human variant FcgR2c is expressed in NK cells exclusively but only in about 20% of the population. Therefore, due to the disparate expression of FcgRs in mouse and human effector cells the assessment of the biological efficacy of human therapeutic antibodies in the mouse xenograft system is of poor predictive value. Because of the intrinsic differences between murine and human effector cells and -mechanisms the obtained data of the xenograft assay is more predictive of the efficacy of TherAbs in mice rather than in humans.

To date, various in vivo models that more closely mimic the human effector cells and—mechanisms have been developed. For example, transgenic mice that express individual human FcgR genes have been generated. However, this mouse model is only partially comparable to that in humans, both in terms of cell distribution and expression levels. Apparently the highly conserved locus of the low affinity FcgR genes (located on chromosome 1 both in men and mice) includes regulatory elements in the intragenic regions that are required for faithful expression of individual FcgR genes and cannot be dissociated from the local genetic context.

Another approach was therefore to replicate the human expression pattern in transgenic mice by the use of transgenic bacterial artificial chromosomes (BACs) bearing up to 200 kb of genomic human DNA. However, in BAC transgenic mice, human and endogenous mouse FcgRs are expressed, leading to abnormally raised overall levels of FcgRs. In addition, the resulting cellular distribution is the sum of murine and human expression patterns.

Therefore there is still a need for an in vivo model that reproduces the expression of human FcgR genes in a non-human animal to improve the predictive potential of the xenograft assay.

The present invention relates to a novel transgenic non-human animal with a humanized low affinity FcgR locus. The non-human animal may be any non-human animal. Preferably, the non-human animal is a mammal, more preferably a rodent such as rat or a mouse, most preferably, the non-human animal is a mouse. In a preferred embodiment, said non-human animal is a mouse.

Provided therein are transgenic non-human animals that comprise a replacement of endogenous low affinity FcgR genes by human low affinity FcgR genes and including non-human animals that are capable of expressing four functional human low affinity FcgR genes, and including non-human animals that do not express endogenous low affinity FcgR genes. In one embodiment said four functional human low-affinity FcgR genes are FCGR2A, FCGR3A, FCGR2C and FCGR3B.

In this novel transgenic non-human animal, the entire low affinity FcgR genes are replaced by the human counterparts. For example in one embodiment the non-human animal is a mouse, wherein the locus encompassing the two murine low affinity receptor genes (Fcgr4, Fcgr3) is replaced with the four human genes (FCGR2A, FCGR3A, FCGR2C, FCGR3B). Thus a mouse line is established capable of expressing the set of human low-affinity FcgR in place of the murine genes and at levels and with cell specificity reminiscent of the human expression. Such a FcgR-humanized mouse line represents an ideal tool for efficacy prediction of therapeutic human antibodies as it combines accurate reproduction of human FcgR expression levels and cell specificity while avoiding cumulative expression of mouse FcgRs and mixed cellular distribution.

In one embodiment the non-human animal expresses human FCGR2A on macrophages and granulocytes, human FCGR3A on NK cells and macrophages, human FCGR3B on granulocytes, both in heterozygous and homozygous targeted mice. In another embodiment said non-human animal does not express endogenous murine low affinity Fcgr4 and Fcgr3 genes.

Since the non-human animal according to the present invention expresses the set of human low-affinity FcgR in place of the endogenous genes and with a cell specificity pattern reminiscent of the human expression, it is useful as an in vivo model to determine antibody ADCC efficacy. Hence in a second object of the invention, said non-human test animal is used as an in vivo model to determine antibody efficacy.

In one embodiment a method for determining the efficacy of an antibody is provided, comprising administering the antibody to be evaluated to a non-human animal with a humanized low affinity FcgR locus according to the present invention. In one embodiment, said method comprises a) providing the non-human animal with a humanized low affinity FcgR locus, b) inoculating a target tumour to said non-human animal and allowing it to grow, c) administering the antibody to be evaluated. The efficacy of the antibody is determined by their capacity to eliminate the tumor or their ability to retard growth of the tumor. Preferably said tumour is a human tumour. In one preferred embodiment said antibody is a human or humanized antibody targeting a tumour cell surface antigen.

The term “replacement” includes wherein a DNA sequence is placed into a genome of a cell of a non-human animal in such a way as to replace a sequence within the genome of the non-human animal with a human sequence.

The term “FcgR” includes a receptor for an Fc, e.g. the Fc portion of an IgG immunoglobulin. The FcgR genes include an α-chain that is expressed on the surface of the cell and serves as a ligand-binding domain, and associates with either a homodimer of the FcRγ-chain or a heterodimer of the FcRγ-chain and the α-chain. There are several different FcgR genes and they can be categorized into low affinity and high affinity types according to preferential binding to IgG in immune complexes. Low affinity FcgR genes in humans include FCGR2A, FCGR3A, FCGR2C, FCGR3B and FCGR2B and within most of these genes naturally occurring genetic differences, or polymorphisms, have been described in human subjects with autoimmune diseases

The term “humanized low affinity FcgR locus” as used herein relates to the part of the genome of a non-human animal encoding the low affinity endogenous FcgR genes that has been replaced with a sequence encoding the human FCGR2A, FCGR3A, FCGR2C and FCGR3B genes and adjacent non-coding regions.

The term “endogenous low affinity FcgR genes” as used herein relates to the low affinity FcgR genes naturally occurring in the genome of the non-human animal. For example the endogenous low-affinity FcgR genes of a mouse are FcgR3 and FcgR4.

The term “wild type” as used herein refers to a non-human animal having a endogenous low affinity FcgR genes and no humanized low affinity FcgR locus.

The term “efficacy of an antibody” as used herein relates to the extent of antibody dependent cell cytotoxicity (ADCC) in a cell. ADCC mediates lysis of a target cell, by an effector cell of the immune system. Therefore the efficacy of an antibody can be determined by its capacity to eliminate a target cell or its ability to retard growth of the target cell. Preferably said target cell is a tumor cell.

Antibodies to be evaluated include, but are not limited to antibodies targeting an epitope of a tumor cell.

Methods of administration of an antibody to be evaluated include, but are not limited to, oral administration and parenteral administration (e.g. intravenous administration, intraperitoneal administration and intranasal administration). The antibody to be evaluated may be administered in combination with a pharmaceutically acceptable conventional excipient (such as carrier and diluent) or additives. Administration of therapeutic antibodies is often accompanied by acute infusion reactions, like skin rush, nausea, anaphylactoid reactions and cytokine release syndroms. Most of these adverse effects involve interaction of the infused antibodies and FcgR's on effector cells like macrophages, NK cells and neutrophils. Thus, thesnon-human animals provided herein are useful as an in vivo model for the assessment of acute adverse effects as provoked by the first infusion of antibodies in humans.

The present invention also relates to descendants of the non-human animals with a humanized low affinity FcgR locus as provided by the invention, obtained by breeding with the same or with another genotype. Preferably, the descendant is obtained by breeding with the same genotype. A further object of the invention is the use of said descendants as an in vivo model for determining the efficacy of an antibody. A further object of the invention is the use of said mice as an in vivo model for assessment of acute infusion reactions as effected by first infusion of therapeutic antibodies and mediated by FcgR's on different effector cells In one embodiment a method for the assessment of the potential safety risk of therapeutic antibodies is provided, comprising administering a therapeutic antibody to the non-human animal of the invention or to its descendant, and evaluating whether acute infusion reactions occur. Acute infusion reactions can be measured by assessing anaphylactoid reactions or cytokine release sydrome.

Furthermore, the present invention relates to a cell line or primary cell culture derived from a non-human animal with a humanized low affinity FcgR locus or its descendants as described above.

In addition, the present invention also provides a tissue or an organ explant or culture thereof, derived from a non-human animal with a humanized low affinity FcgR locus or its descendants as described above.

The present invention also provides a tissue or cell extract derived a non-human animal with a humanized low affinity FcgR locus or its descendants as described above.

In another embodiment of the invention, above-mentioned cell line or primary cell culture, tissue or an organ explant or culture thereof, tissue or cell extract derived from a non-human animal with a humanized low affinity FcgR locus or its descendants is used as an in vivo model to determine the efficacy of an antibody.

“Non-human animal” as described herein refers to any animal that is not a human. Preferably, the non-human animal is a mammal, more preferably a rodent such as rat or a mouse, most preferably, the non-human animal is a mouse.

### SHORT DESCRIPTION OF THE FIGURES

FIG. 1—Schematic representation of the RMGR process leading to replacement of the two murine low-affinity FcgR genes, Fcgr4 and Fcgr3 (upper line), by the four human low-affinity FcgR genes, FCGR2A, FCGR3A, FCGR2C and FCGR3B, placed in the BAC targeting construct (middle line). The mutated, “humanized” allele resulting from the Cre-mediated recombination is depicted in the lower line.

FIG. 2—(A) Schematic representation of the mouse low affinity Fcgr locus upon replacement of a 54 kb long region by 160 kb of human DNA containing the entire set of human low affinity FcR genes through Cre-aided recombination at the indicated LoxP and Lox511 elements (colored triangles). The position of the oligonucleotides used in the detection PCR assays is indicated by short arrows pointing in their corresponding orientation. (B) The DNA fragments amplified from individual mouse biopsies in the PCR assays indicated (5′ end: ˜5 kb; 3′ end: 1.2 kb) is shown upon separation on 1% agarose gels. Tm indicates mutant mice, wt stand for wild type mice; M, marker DNA.

FIG. 3—(A) The FCGR-humanized locus is depicted schematically to indicate the position of the primers used for PCR amplification of the individual human FCGR genes, as in

FIG. 2 but with abbreviated names. The genes encoding FCGR2A, FCGR3A, FCGR2C and FCGR3B were amplified with the primer pairs 1+2, 3+4, 5+6 and 7+8, respectively. The position of the SalI restriction cut is indicated by a vertical arrow. (B) The agarose gel displays the DNA fragments amplified from biopsy DNA taken from a mouse heterozygous for the replacement mutation. Specific primers were used to amplify the human genes FCGR2A (2A: 12.9 kb), FCGR3A (3A: 9.6 kb), FCGR2C (2C: 18.5 kb) and FCGR3B (3B: 9.6 kb). X and IV indicate the DNA molecular weight markers (in kb) used (Roche Diagnostics). (C) The specificity of the two 9.6 PCR fragments amplified with primers specific for 3A and 3B PCR was tested by SalI digestion: while the 3A fragment is cleaved with SalI into a 8 kb and a 1.65 kb fragment, the 9.6 kb PCR fragment amplified from the FCGR3B gene (3B) is resistant to SalI digestion, thus demonstrating that the amplified fragments correspond to the two different genes.

FIG. 4—(A) Expression of human FCGR3A (CD16) in peripheral blood cells of gene-targeted human Human FCGR2A/3A/2C/3B Heterozygous mice and wild type mice was monitored by FACS analysis using a FACS Canto device after staining with the antibodies indicated: Expression was clearly detected in F4/80+ monocytes, NK1.1+ natural killer cells (both within the lymphoid gating) and in Gr-1+ granulocytes, as detected within the myeloid gating. (B) Expression of human FCGR2A (CD32) in peripheral blood cells of gene-targeted Human FCGR2A/3A/2C/3B Heterozygous mice and wild type mice, as in (A): Expression was evident in F4/80+ monocytes and in Gr-1+ granulocytes but was absent in NK1.1+ natural killer cells, as expected for human CD32.

FIG. 5—(A) Expression of human FCGR3A (CD16) in peripheral blood cells of gene-targeted human Human FCGR2A/3A/2C/3B Homozygous mice and wild type mice was monitored by FACS analysis using a FACS Canto device after staining with the antibodies indicated: Expression was clearly detected in F4/80+ monocytes, NK1.1+ natural killer cells (both within the lymphoid gating) and in Gr-1+ granulocytes, as detected within the myeloid gating. (B) Expression of human FCGR2A (CD32) in peripheral blood cells of gene-targeted Human FCGR2A/3A/2C/3B Homozygous mice and wild type mice, as in (A): Expression was evident in F4/80+ monocytes and in Gr-1+ granulocytes but was absent in NK1.1+ natural killer cells, as expected for human CD32.

FIG. 6—(A) The amount of cells bearing surface human FcγRIII or FcγRII within the indicated cell population in peripheral blood of gene-targeted human Human FCGR2A/3A/2C/3B Homozygous mice is shown. The histograms display the amount of cells positive for human FcγR's in homozygous gene-targeted mice (black line) in comparison with wild type mice (grey line), as detected in F4/80+ monocytes, NK1.1+ natural killer cells (both within the lymphoid gating) and in Gr-1+ granulocytes (within the myeloid gating).

TABLE 1

| Mice: Human | | Monocytes | NK cells | Granulocytes |
|---|---|---|---|---|
| FCGR2A/3A/2C/3B | | (F4/80) | (NK1.1) | (Gr-1) |
| CD16 | Het | 58 | 30 | 94 |
| | Hom | 59 | 31 | 98 |
| CD32 | Het | 86 | 0 | 97 |
| | Hom | 81 | 0 | 98 |

The table summarizes the percentages of peripheral blood cells expressing surface human FcγRIII (CD16) or FcγRII (CD32) within selected populations of F4/80+ monocytes; NK1.1+ Natural Killer cells or Gr-1+ Granulocytes, in Human FCGR2A/3A/2C/3B heterozygous (Het) or homozygous (Hom) mice.

### EXAMPLES

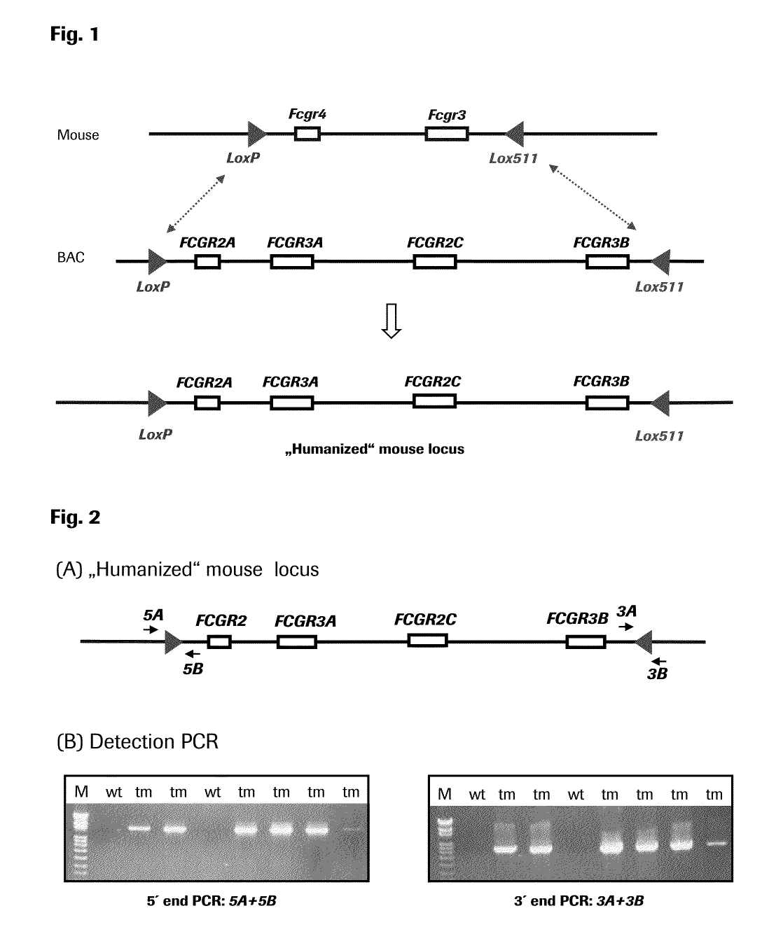

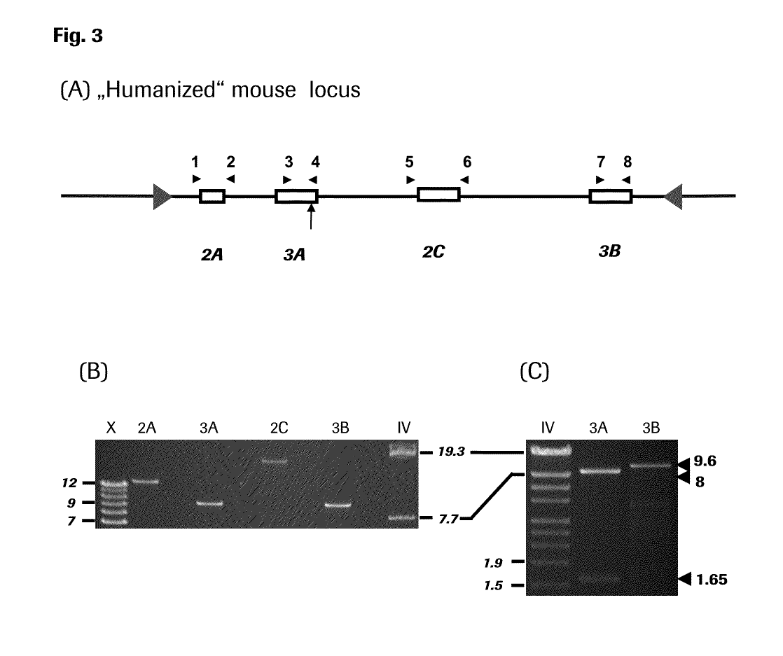

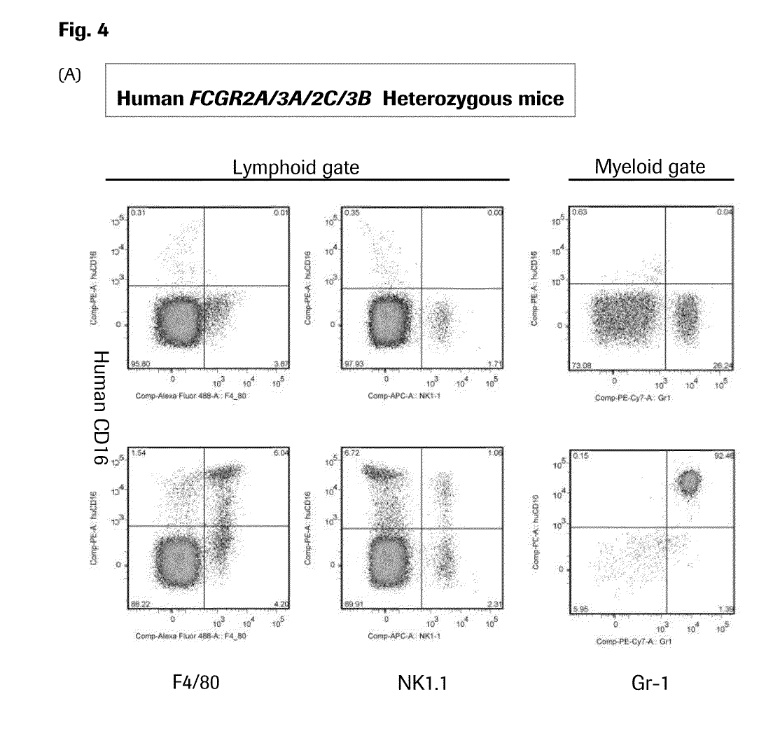

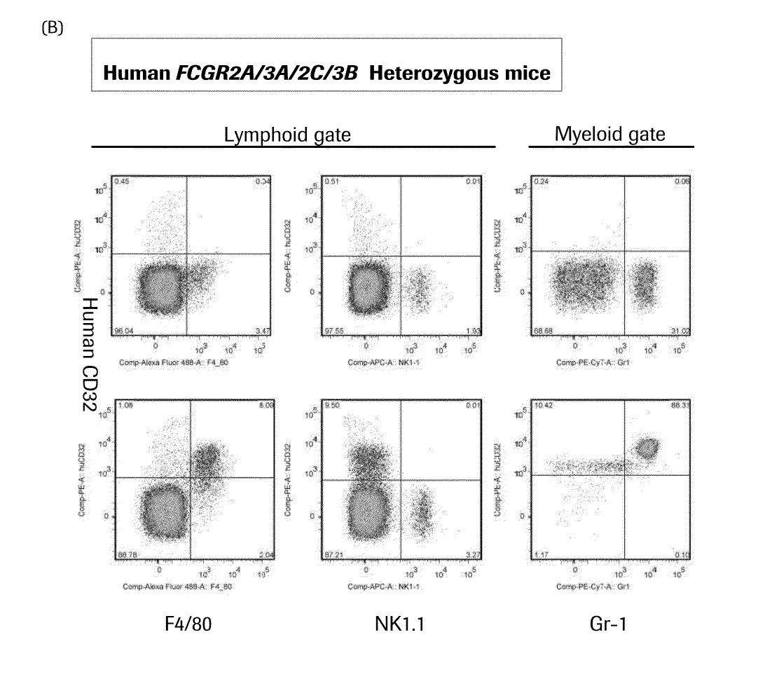

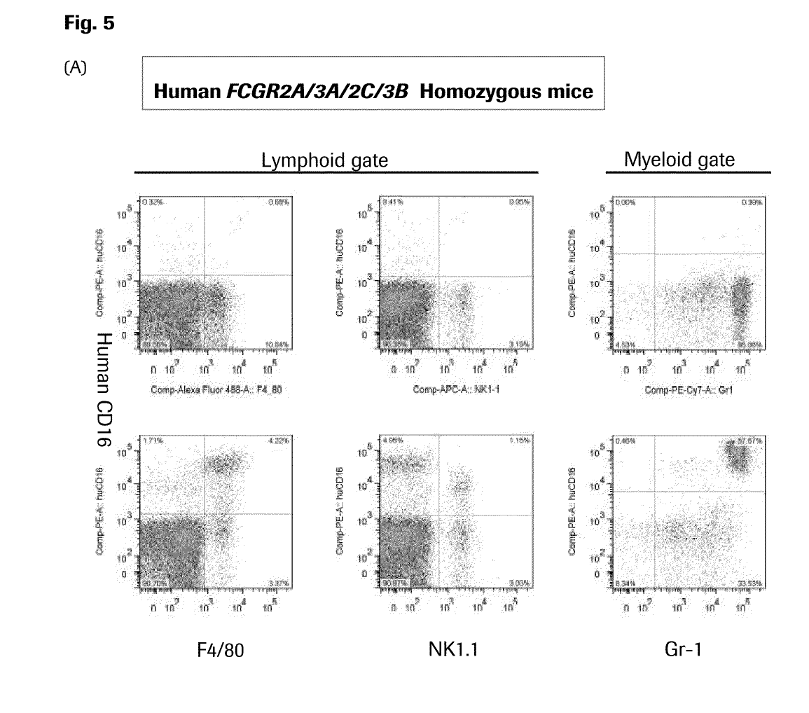

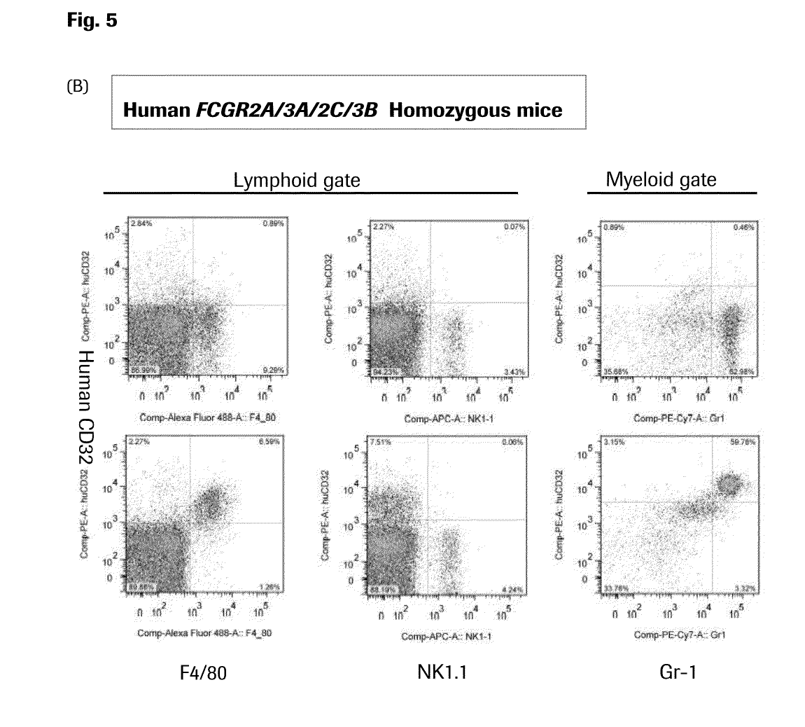

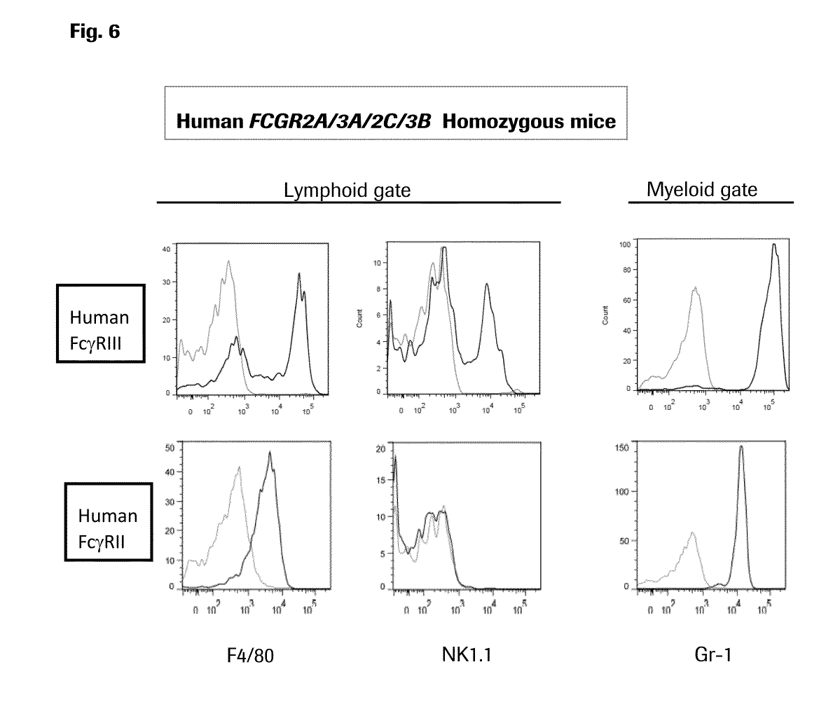

The entire locus encompassing the two murine low affinity receptor genes (Fcgr4, Fcgr3) was replaced with the four human genes (FCGR2A, FCGR3A, FCGR2C, FCGR3B) via recombinase mediated gene replacement (RMGR). Using this technique 54 kb of the mouse genome encompassing Fcgr4, Fcgr3 and adjacent non-coding DNA regions are first flanked with two incompatible Lox elements (LoxP and Lox511) via consecutive homologous gene targeting in mouse ES cells. In parallel a BAC construct is prepared containing 160 kb of human genomic DNA encompassing the genes FCGR2A, FCGR3A, FCGR2C, FCGR3B and adjacent non-coding regions, also flanked by LoxP and Lox511. With the later DNA construct, the Cre-recombinase mediated exchange of the mouse region by the human DNA will then generate mutant ES cells bearing the four human low-affinity FcgR genes instead of their two murine counterparts and located at their natural position of the mouse genome (see FIGS. 1-3). Upon microinjection of the mutated ES cells into recipient mouse blastocysts chimeric mice are generated. Upon transmission of the mutation onto the next generation, a mouse line is established capable of expressing the set activating human low-affinity FcgR's in place of the two murine counterparts and with a cell specificity pattern that mirrors that of human cells (FIGS. 4-6 and Table 1). Such a FcgR-“humanized” mouse line represent an ideal tool for efficacy prediction of therapeutic human antibodies as it combines accurate reproduction of human FcgR expression levels and cell specificity while avoiding cumulative expression of mouse FcgRs and mixed cellular distribution.

The present invention describes the production of FcgR-humanized mice via RMGR expressing the human FcgR genes according to the typical human cell specificity. These mice, designated henceforth as Human FCGR2A/3A/2C/3B are suitable for the predictive description of the in vivo ADCC potential of therapeutic antibodies and thus the early functional selection among series of therapeutic candidates.

1. A non-human transgenic animal comprising a humanized low affinity FcgR locus.

2. The non-human animal of claim 1, wherein the endogenous low affinity FcgR genes are completely replaced by the human low affinity FcgR genes.

3. The non-human animal of claim 1 or 2, wherein the non-human animal is a mammal.

4. The non-human animal of claim 3, wherein the non-human animal is a rodent.

5. The non-human animal of claim 4, wherein the non-human animal is a mouse.

6. A descendant of the non-human animal of claims 1 to 5, obtained by breeding with animals of the same or another genotype.

7. A cell line or primary cell culture derived from the non-human animal of claims 1 to 5 or from the descendant of claim 6.

8. A tissue or an organ explant or culture thereof derived from the non-human animal of claims 1 to 5, or from the descendant of claim 6.

9. Use of the non-human animal of any of claims 1 to 5, the descendant of claim 6, the cell line or primary cell culture of claim 7 or the tissue or organ explant of claim 8 for determination of the efficacy of antibodies.

10. A method for determining the efficacy of an antibody, comprising inoculating a tumor cell or tumor into the non-human animal of any of claims 1 to 5 or to its descendant of claim 6, and administering the antibody to said non-human animal or to its descendant.

11. A method for the assessment of the potential safety risk of a therapeutic antibody, comprising administering a therapeutic antibody to the non-human animal of any of claims 1 to 5 or to its descendant of claim 6, and evaluating whether any acute infusion reactions occur.

12. The non-human animal, the uses and methods essentially as herein described.
